(19) United States
(12) Patent Application Publication
(10) Pub. No.: US 2007/0219275 A1
(43) Pub. Date: Sep. 20, 2007

(54) Use of Benzophenone Uv Filters for Preventing Tanning

(76) Inventor: Werner Baschong, Basel (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/593,521

(22) PCT Filed: Mar. 23, 2005
(86) PCT No.: PCT/EP05/51325

§ 371 Date: Sep. 20, 2006

(30) Foreign Application Priority Data
Apr. 2, 2004 (EP) 04101375.6

(51) Int. Cl. A61K 31/12

(52) U.S. Cl. 514/678

(57) ABSTRACT

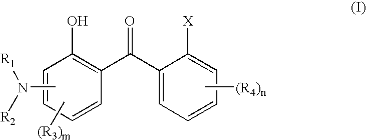

The invention describes the use of organic UV filters of formula (I) wherein R, and R₂, 'independently from each other are hydrogen, C₁-C₂, alkyl, C₂-C₁₀ alkenyl, C3 C,ₒcycloalkyl, C₃-C₁₀cycloalkenyl, or R, and. R₂ togethér with the nitrogen atom to which they are bonded can form a 5- or 6-membered ring; R₃ and R₄, independently from each other are C₁-C₂₀alkyl, C2 C,ₒalkenyl, C₃-C1₂cycloalkyl, C₃-C₁₀cycloalkenyl, C₁-C₁₂alkoxy, C,-C₂₀alkoxycarbonyl, C₁-C,_{z}alkylamino, C₁-C₁₂dialkylamino, aryl, heteroaryl, optionally substituted, substituents which confer solubility in water, chosen from the group consisting of a nitrile group, carboxylate, sulfonate or ammonium radicals; X is hydrogen, COOR₅, CONR₆R₇; R₅, Rₛ and R₇, independently from each other are hydrogen, C₁-C₂₀alkyl, C₂-C₁₀alkenyl, C₃-C,₀cycloalkyl, C3 C₁₀cycloalkenyl, (Y-0), -Z-aryl; Y is —(CH₂)2, —(CH2)3, —(CH₂)₄—, —CH(CH₃)—CH2; Z is —CH2 CH₃ᵢ —CH₂—CH,CH₃, —CH₂ CH₂ CH2 CH₃, —CH(CH₃)—CH,; m is from 0 to 3; n. is from 0 to 4; o is from 1 to 20; for preventing tanning human skin, and their use in cosmetic and pharmaceutical formulations, as anti-wrinkling agents, skin-firming agents and as anti-skin lifting agents.

The present invention relates to the use of specific organic UV filters for preventing tanning of the human skin and to their use in cosmetic formulations.

The topical application of UV-filters in order to prevent or reduce sunburn, premature aging and pathologic alterations of the skin has become well established for Caucasians where sensitive skin types are abundant and social cultural habits propagate body exposure and a well tanned skin complexion.

But also Non-Caucasians are prone to UV-sensitive pigmentation disorders such as vitiligo or melasma. Such disturbances can manifest much more in a colored complexion and may even reach the level of disfigurement. This is one of the reasons why the beauty ideal of many colored persons and especially those of Asian origin traditionally avoid skin exposure to the sun and show only light pigmentation.

Since dressing habits and outdoor-activities are changing towards more sun exposure, daily cosmetics containing sun protection factors are becoming increasingly popular. Nonetheless, to our knowledge only few clinical reports focus on UV-induced skin damage in Non-Caucasians and most of them concentrate on the impact of UV-light rather than on the efficacy of prevention.

Particularly in Asian countries, there is therefore a great interest in light protection filters or mixtures of light protection filters which preserve the color of the skin following solar irradiation and, moreover, are able to impart a lighter appearance to the skin.

The object of the present invention is therefore to find organic UV filters, which prevent tanning of the skin and at the same time, in combination with pigment-regulators are able to lighten the skin.

Surprisingly, we have now found that specific benzophenone organic UV filters can achieve this object.

The present invention therefore provides for the use of (a) UV filters selected from the compounds of formula

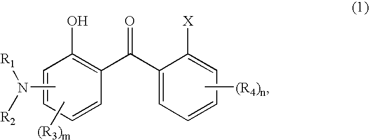

R₁ and R₂, independently from each other are hydrogen; C₁-C₂₀alkyl; C₂ -C₁₀alkenyl; C₃-C₁₀cycloalkyl; C₃-C₁₀cycloalkenyl; or R₁ and R₂ together with the nitrogen atom to which they are bonded can form a 5- or 6-membered ring;

R₃ and R_{4,} independently from each other are C₁-C₂₀alkyl; C₂-C₁₀alkenyl; C₃-C₁₀-cycloalkyl; C₃-Clocycloalkenyl; C₁-C₁₂alkoxy; C₁-C₂₀alkoxycarbonyl; C₁-C₁₂alkylamino; C₁-C₁₂dialkylamino; C₆-C₁₀aryl; C₆-C₁₀heteroaryl, optionally substituted, substituents which confer solubility in water, chosen from the group consisting of a nitrile group, carboxylate, sulfonate or ammonium radicals;

X is hydrogen; COOR₅; CONR₆R₇;

R₆ and R₇, independently from each other are hydrogen; C₁-C₂₀alkyl; C₂-C₁₀alkenyl; C₃-Clocycloalkyl; C₃-C₁₀cycloalkenyl; or (Y—O)ₒ-Z-aryl;

Y is —(CH₂)₂—; —(CH₂)₃; —(CH₂)₄—; —CH(CH₃)—CH₂—;

Z is —CH₂—CH₃; —CH₂—CH₂—CH₃; —CH₂—CH₂—CH₂—CH₃; —CH(CH₃)—CH₃;

m is a number from 0 to 3;

n is a number from 0 to 4; and

o is a number from 1 to 20;

as organic UV filters for preventing tanning human skin.

Alkyl radicals R₁ to R₇ which may be mentioned are branched or unbranched C₁-C₂₀alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, 2-ethylhexyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

Alkenyl radicals R₁ to R₇ which may be mentioned are branched or unbranched C₂-C₁₀alkenyl chains, preferably vinyl, propenyl, isopropenyl, 1-butenyl, 2-butenyl, 1-pentenyl, 2-pentenyl, 2-methyl-1-butenyl, 2-methyl-2-butenyl, 3-methyl-1-butenyl, 1-hexenyl, 2-hexenyl, 1-heptenyl, 2-heptenyl, 1-octenyl or 2-octenyl.

Cycloalkyl radicals which may be mentioned for R₁ to R₇ are preferably branched or unbranched C₃-C₁₀-cycloalkyl chains such as cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentylcyclopropyl, 1-methyl-1-butylcyclopropyl, 1,2-dimethylcyclopropyl, 1-methyl-2-ethylcyclopropyl, cyclooctyl, cyclononyl or cyclodecyl.

Cycloalkenyl radicals which may be mentioned for R₁ to R₇ are preferably branched or unbranched C₃-C₁₀cycloalkenyl chains with one or more double bonds such as cyclopropenyl, cyclobutenyl, cyclopentenyl, cyclopentadienyl, cyclohexenyl, 1,3-cyclohexadienyl, 1,4-cyclohexadienyl, cycloheptenyl, cycloheptatrienyl, cyclooctenyl, 1,5-cyclooctadienyl, cyclooctatetraenyl, cyclononenyl or cyclodecenyl.

The cycloalkenyl and cycloalkyl radicals may be unsubstituted or substituted by one or more, e.g. 1 to 3, radicals such as halogen, e.g. fluorine, chlorine or bromine, cyano, nitro, amino, C₁-C₄-alkylamino, C₁-C₄-dialkylamino, hydroxyl, C₁-C₄-alkyl, C₁-C₄-alkoxy or other radicals, or contain 1 to 3 heteroatoms such as sulfur, nitrogen, whose free valences can be saturated by hydrogen or C₁-C₄-alkyl, or oxygen in the ring.

Suitable alkoxy radicals for R₃ and R₄ are those having 1 to 12 carbon atoms, preferably having 1 to 8 carbon atoms.

Examples which may be mentioned are: methoxy, ethoxy, isopropoxy, n-propoxy, 1-methylpropoxy, n-butoxy, n-pentyloxy, 2-methylpropoxy, 3-methylbutoxy, 1,1-dimethylpropoxy, 2,2dimethylpropoxy, hexyloxy, 1-methyl-1-ethylpropoxy, heptyloxy, octyloxy or 2-ethylhexyloxy.

Examples of alkoxycarbonyl radicals for R₃ and R₄ are esters containing the abovementioned alkoxy radicals or radicals derived from higher alcohols, e.g. having up to 20 carbon atoms, such as iso-C₁₅ alcohol.

Suitable mono- or dialkylamino radicals for R₃ and R₄ are those containing alkyl radicals having 1 to 12 carbon atoms, such as methyl, n-propyl, n-butyl, 2-methylpropyl, 1,1-dimethylpropyl, hexyl, heptyl, 2-ethylhexyl, isopropyl, 1-methylpropyl, n-pentyl, 3-methylbutyl, 2,2-dimethylpropyl, 1-methyl-1-ethylpropyl and octyl.

Aryl means aromatic rings or ring systems having 6 to 18 carbon atoms in the ring system, for example phenyl or naphthyl, each of which may be unsubstituted or substituted by one or more radicals such as halogen, e.g. fluorine, chlorine or bromine, cyano, nitro, amino, C₁-C₄-alkylamino, C₁-C₄-dialkylamino, hydroxyl, C₁-C₄-alkyl, C₁-C₄-alkoxy or other radicals. Unsubstituted or substituted phenyl, methoxyphenyl and naphthyl are preferred.

Heteroaryl radicals are advantageously simple or fused aromatic ring systems having one or more heteroaromatic 3- to 7-membered rings. Heteroatoms which may be present in the ring or ring system are one or more nitrogen, sulfur and/or oxygen atoms. Hydrophilic radicals, i.e. those making it possible for the compounds of the formula (1) to dissolve in water. R₃ and R₄ are, for example, the nitrile group and carboxyl and sulfoxy radicals and, in particular, their salts with any physiologically tolerated cations, such as the alkali metal salts or such as the trialkylammonium salts, such as tri(hydroxyalkyl)ammonium salts or the 2-methyl-1-propanol-2-ammonium salts. Also suitable are ammonium radicals, especially alkylammonium radicals, with any physiologically tolerated anions.

The substituents R₁ and R₂ may, together with the nitrogen atom to which they are bonded, form a 5- or 6-membered ring, for example a pyrrolidine or piperidine ring.

The amino group can be in the ortho, meta or para position relative to the carbonyl group. The para position is preferred.

Preferably compounds of formula 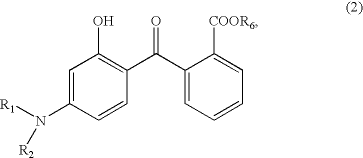
are used, wherein R₁ and R₂ independently from each other are hydrogen, C₁-C₁₂alkyl; or R₁ and R.₂ together with the nitrogen atom to which they are bonded form a 5- or 6-membered ring; and

R₅ is hydrogen, C₁-C₁₂alkyl, C₃-C₆cycloalkyl.

Preferably compounds of formula (1) are used, wherein R₁ and R₂, independently from each other are C₁-C₅alkyl; and

R₅ is C₁-C₁₂alkyl; and more preferably compounds of formula (1) or (2), wherein

R₁ and R₂, independently from each other are C₁-C₅alkyl; and

R₅ is C₁-C₁₂alkyl.

Compounds of formula (1) or (2), wherein R₁ and R₂ have the same meaning, are of most preferred interest.

Most preferably the compound of formula 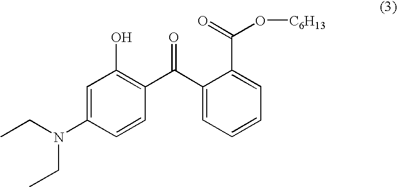
is used.

The compounds of formula (1) are suitable as active ingredients in cosmetic preparations. The compounds can preferably be used in dissolved form.

The cosmetic formulations or pharmaceutical compositions according to the present invention may additionally contain one or more than one further UV filter (b).

The additional UV filters (b) are selected from p-aminobenzoic acid derivatives, salicylic acid derivatives, benzophenone derivatives different from those of formula (1), dibenzoylmethane derivatives, diphenylacrylates, 3-imidazol4-ylacrylic acid and esters; benzofuran derivatives, polymeric UV absorbers, cinnamic acid derivatives, camphor derivatives, hydroxyphenyltriazine compounds, benzotriazole compounds, trianilino-s-triazine derivatives, 2-phenylbenzimidazole-5-sulfonic acid and salts thereof, menthyl o-aminobenzoates, physical coated or non-coated sunscreens, perfluoroalcohol phosphate, aminohydroxy-benzophenone derivatives and phenyl-benzimidazole derivatives.

Preferably, the following UV filter combinations are of special interest:
UV-filter combinations (A) comprising

(a₁) at least one UV-filter of formula (1) and

(a₂) at least one aminobenzophenone derivative
UV-filter combinations (B) comprising

(b₁) at least one UV-filter of formula (1), preferably the compound of formula (3); and

(b₂) benzoic acid,4,4′-[[6-[[4-[[(1,1 -dimethylethyl)amino]carbonyl]phenyl]amino]1,3,5-triazine-2,4-diyl]diimino]bis-,bis(2-ethylhexyl)ester; diethyihexyl butamido triazone (Uvasorb HEB);
UV-filter combinations (C) comprising

(c₁) at least one UV filter of formula (1); and

(c2) at least one benzotriazole derivative of formula 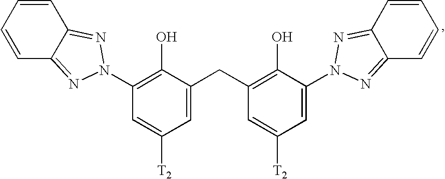
wherein

T₂ is C₁-C₁₀alkyl or phenyl-substituted C₁-C₄alkyl;
UV-filter combinations (D) comprising

(d₁) at least one UV-filter of formula (1); and

(d₂) at least hydroxyphenyltriazine compound

Most preferred are UV-filter combinations (D1) comprising

(d₃) the compound of formula (3); and

(d₄) the compound of formula 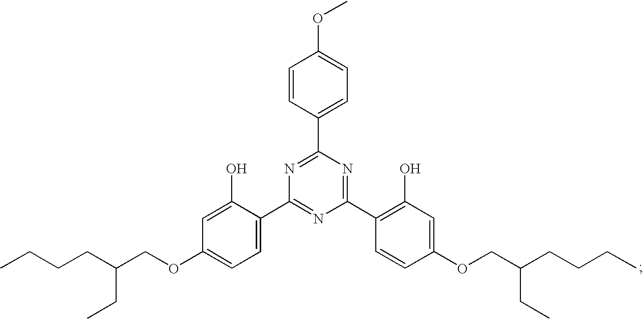
UV-filter combinations (F) comprising

(f₁) at least one UV-filter of formula (1); and

(f₂) at least one dibenzoylmethane derivative

Most preferred are UV-filter combinations (F1) comprising

(f3) the compound of formula (3); and

(f₄) 1-[4-(1,1-dimethylethyl)phenyl]-3-(4-methoxyphenyl)propane-1,3-dione (Avobenzone);
UV-filter combinations (G) comprising

(g₁) at least one UV-filter of formula (1), preferably the compound of formula (3); and

(g₂) disodium phenyl dibenzimidazole tetrasulfonate (Heliopan AP).
UV-filter combinations (H) comprising

(h₁) at least one symmetrical triazine derivatives of formula (1); and

(h₂) benzoxazole-substituted triazines of formula 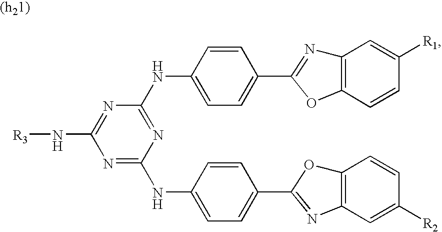
R₁, R₂ and R₃ independently from each other are branched or unbranched C₁-C₁₂alkyl.

Furthermore, UV filter combination (H2) comprising

(h5) the compound of formula (3); and

(h₆) at least one of the compound of formula (h₂1), wherein

(h₆₁) R₁ and R₂ are tert.amyl; and R₃ is tert.butyl; or wherein

(h₆₂) R₁ and R₂ are tert.butyl and R₃ is tert.octyl; or wherein

(h₆₃) R₁ and R₂ are tert.butyl; and R₃ is 2-ethylhexyl; or wherein

(h₆₄) R₁ and R₂ are tert.amyl; and R₃ is 2-ethylhexyl; are of preferred interest.
UV-filter combinations (I) comprising

(i₁) at least one UV-filter of formula (1), preferably the compound of formula (3); and

(i₂) 2-(2H-benzotriazol-2-yl)-4-methyl-6-[2-methyl-3-[1,3,3,3-tetramethyl-1-[(trimethyl-silyl)oxy]disiloxanyl]propyl]-; (CAS-No. 155633-54-8; Drometrizole Trisiloxane; Mexoryl XL);
UV-filter combinations (K) comprising

(k₁) at least one UV-filter of formula (1), preferably the compound of formula (3); and

(k₂) siloxanes and silicones, di-Me, 1-[[4-[3-ethoxy-2-(ethoxycarbonyl)-3-oxo-1-propenyl]phenoxy]methyl]ethenyl Me, 3-[4-[3-ethoxy-2-(ethoxycarbonyl)-3-oxo-1-propenyl]-phenoxy]-1-propenyl Me, Me hydrogen (Dimethicodiethylbenzalmalonate; CAS-No. 207574-74-1);
UV-filter combinations (L) comprising

(l₁) at least one UV-filter of formula (1), preferably the compound of formula (3); and

(l₂) (+/−)-1,7,7-trimethyl-3-[(4-methylphenyl)methylene]bicyclo[2.2.1]heptan-2-one; p-methyl benzylidene camphor;
UV-filter combinations (M) comprising

(m₁)at least one UV-filter of formula (1), preferably the compound of formula (3); and

(m₂) 1-(2-oxoborn-3-ylidene)toluene4-sulphonic acid and its salts (Mexoryl SL);
UV-filter combinations (N) comprising

(n₁) at least one UV-filter of formula (1), preferably the compound of formula (3); and

(n₂) methyl N,N,N-trimethyl-4-[(4,7,7-trimethyl-3-oxobicyclo[2,2,1]hept-2-ylidene)methyl]-anilinium sulphate (Mexoryl SO);
UV-filter combinations (O) comprising

(o₁) at least one UV-filter of formula (1), preferably the compound of formula (3); and

(o₂) 2-ethylhexyl 2-cyano,3,3-diphenylacrylate (Octocrylene);
UV-filter combinations (P) comprising

(p₁) at least one UV-filter of formula (1), preferably the micronized compound of formula (3); and

(p₂) 2-ethylhexyl 4-methoxycinnamate (octyl methoxy cinnamate);
UV-filter combinations (Q) comprising

(q₁) at least one UV-filter of formula (1), preferably the compound of formula (3); and

(q₂) benzoic acid, 4,4′,4″-(1,3,5-triazine-2,4,6-triyltriimino)tris-,tris(2-ethylhexyl)ester; 2,4,6-Trianilino-(p-carbo-2′-ethylhexyl-1′-oxi)-1,3,5-triazine (Ethylhexyl Triazone);
UV-filter combinations (R) comprising

(r₁) at least one UV-filter of formula (1), preferably the compound of formula (3); and

(r₂) 2-phenyl-1H-benzimidazole-5-sulphonic acid (Phenylbenzimidazol sulfonic Acid);

Cosmetic compositions comprising a UV filter according to component (a) and optionally of component (b) are useful anti-tanning agents.

Cosmetic or pharmaceutical preparations contain from 0.05-40% by weight, based on the total weight of the composition, of a UV filter according to component (a) and optionally of component (b).

Preference is given to the use of mixing ratios of the UV absorber of formula (1) (component (a)) and optionally further UV filters (component (b)) (as for example described in Table 1-3) from 1:99 to 99:1, preferably from 1:95 to 95:1 and most preferably from 10:90 to 90:10, based on weight. Of special interest are mixing ratios of from 20:80 to 80:20, preferably from 40:60 to 60:40 and most preferably approximately 50:50. Such mixtures can be used, inter alia, to improve the solubility or to increase UV absorption.

The UV filters of component (b) according to the present invention can be used either in the dissolved state (soluble organic filters, solubilized organic filters) or in the micronised state (nanoscalar organic filters, particulate organic filters, UV-absorber pigments).

Any known process suitable for the preparation of microparticles can be used for the preparation of the micronised UV absorbers, like wet-milling, wet-kneading, spray-drying from a suitable solvent, by the expansion according to the RESS process (Rapid Expansion of Supercritical Solutions) of supercritical fluids (e.g. CO₂) in which the UV filter or filters is/are dissolved, or the expansion of fluid carbon dioxide together with a solution of one or more UV filters in a suitable organic solvent; or by reprecipitation from suitable solvents, including supercritical fluids (GASR process=Gas Anti-Solvent Recrystallisation/PCA process=Precipitation with Compressed Anti-solvents).

The micronised UV absorbers so obtained usually have an average particle size from 0.02 to 2, preferably from 0.03 to 1.5, and more especially from 0.05 to 1.0 micrometer.

The UV absorbers according to the present invention can also be used as dry substrates in powder form.

The UV absorbers according to the present invention can also be used in specific carriers for cosmetics, for example in solid lipid nanoparticles (SLN) or in inert sol-gel microcapsules wherein the UV absorbers are encapsulated (Pharmazie, 2001 (56), p. 783-786). Lipid nanoparticles (CLN,=Crystalline Lipid Nanoparticles) as described in Internat. J. Pharmaceutics, 2002, 242, P. 373-375 can be used as active carrier for UV filter according to the invention.

The cosmetic composition used according to the present invention may additionally contain one or more than one further UV filter.

The UV filters according to component (a) and (b) can be prepared for their use in cosmetic preparations by physically mixing the UV filter(s) with the adjuvant using customary methods, for example by simply stirring together the individual components, especially by making use of the dissolution properties of already known cosmetic UV fitlers, like octyl methoxy cinnamate, salicylic acid isooctyl ester, etc. The UV filter can be used, for example, without further treatment, or in the micronised state, or in the form of a powder.

As component (c) cosmetic and/or pharmaceutical active substances can be used.

Examples of active ingredients which can be used and which may be mentioned are:
active ingredients for antimicrobial finishing and simultaneous antiinflammatory action;
antiinflammatory active ingredients, for example farnesol, panthenol or avocado oil;
active ingredients having a deodorant or antiperspirant action, for example Zn ricinoleates and alkyl citrates,
undecylenic acid and derivatives thereof (e.g. diethanolamides)
zinc undecylate;
pyrithiones, for example sodium pyrithione;
fused-in fragrances or fragrance mixtures, for example menthol, geraniol etc., which impart a permanent odour which is uniform in intensity to these micropigments and the formulations which comprise them.
biogenic active ingredients like tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, plant extracts and vitamin complexes.
antioxidants:
Amino acids (glycine, histidine, tyrosine, tryptophane) and derivatives thereof,
imidazole, like urocanine acid and dervatives thereof,
peptides likeD, L-carnosine, D-carnosine, L-carnosine and derivatives therof (like anserine),
carotinoides, carotenes like α-carotin, β-carotin, lycopin and derivatives thereof,
chlorogene acid and derivatives thereof,
liponic acid and derivatives thereof like dihydroliponic acid,
aurothioglycose, propylthiouracil and other thioles like thioredoxin, glutathion, cystein, cystin, Cystamine and their glycosyl-, N-acetyl-, methyl-, ethyl-, propyl-, amyl-, butyl- and lauryl-, palmitoyl-, oleyl-, a-linoleyl-, cholesteryl- and glyceryl esters and salts thereof,
dilaurylthiodipropionate, distearylthiodipropionate, thiodipropionic acid and derivatives thereof (ester, ether, -peptides, lipids, nucleotides, nucleosides and salts thereof),
sulfoximine compounds like buthioninsulfoximine, homocysteinsulfoximine, butionin-sulfon, penta-, hexa-, heptathioninsulfoximine,
(metal)-chelating agents like a-hydroxy fatty acids, palmitic acids, phytinic acid, lacto-ferrine, α-hydroxy acids like citric acid, lactic acid, gallic extracts like bilirubin, biliverdin, EDTA, EGTA and derivatives thereof, unsaturated fatty acids and derivatives thereof like α-linolene acid, linolic acid, oil acid), folic acid and derivatives thereof, ubichinone and ubichinol and derivatives thereof, tocopherols and derivatives thereof like vitamin-E-acetate), vitamin A and derivatives thereof like vitamin-A-palmitate and coniferylbenzoate of benzoe resin, rutinic acid and derivatives thereof, α-glycosylrutin, ferulaacid, furfurylidenglucitol, carnosin, butylhydroxytolune, butylhydroxyanisol, nordihydroguajakresin acid, nordihydroguajaretacid, trihydroxybutyrophenone, urea acid and derivatives thereof, mannose and derivatives thereof, superoxid-Dismutase, zinc and derivatives thereof like. ZnO or ZnSO₄), selen und derivatives thereof like selen-methionin, stilbene and derivatives thereof like stilbeneoxide, trans-stilbeneoxide and derivatives thereof.
HALS (=“Hindered Amine Light Stabilizers”) compounds may also be mentioned. Further synthetic and natural antioxidants are listed e.g. in patent WO 0025731: Structures 1-3 (page 2), structure 4 (page 6), structures 5-6 (page 7) and compounds 7-33 (page 8-14).

As component (d) pigment regulators, preferably skin whitening agents, may additionally be used.

Useful representatives for component (d) are the following classes of substances:

1. γ-pyron derivates according to formula 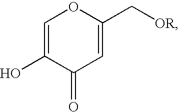
wherein R is hydrogen (=kojic acid; 5-hydroxy-2-hydroxymethyl-4H-pyran-4-on); or the radical of formula

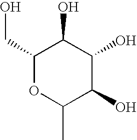

2. Hydrochinon, also as glycosides and hydrochinon derivatives as glycosides, like 4-hydroxyphenyl-D-glucopyranoside (=α-, or β-Arbutin) of formula 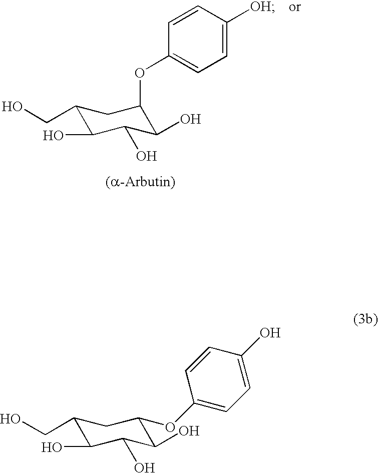
(β-Arbutin); 4-methoxyphenethylmethylether-D-glucopyranoside; 1,5,9,13-tetramethyl-4,8,12-tetradecatrienyl (9CI); 5,9,13-pentadecatriene-2-ol, 6,10,14-trimethyl-(9CI); 1,5,9,1 3-tetramethyltetradecyl-D-glucopyranoside.

3. Resorcin derivatives like glabridin (1,3-benzenediol, 4-[(3R)-3,4-dihydro-8,8-dimethyl-2H,8H-benzo[1,2-b:3,4-b′]dipyran-3-yl]-) or 4-butylresorcinol (=rucinol); 2,4-di-hydroxybenzophenones and isomeric benzophenones;

4. Glycines, L-α-glutamyl-L-cysteinyl- (=glutathion); cetylcystein; oligopeptides;

5. Alkyldicarboxclic acids, like azelain acid (nonandicarboxylic acid) and its mono- and diester;

6. 1,2-dihydroxyphenyl derivatives, like 4-(3,4-dihydroxyphenyl)butan-2-ol; 4-hydroxy-3-methoxybenzylacetone (=gingerone); 4H-1-benzopyran-4-one, 2-(3,4-dihydroxy-phenyl)-3,5,7-trihydroxy-(=quercitin), of formula

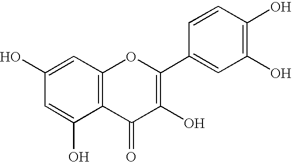

7. Urea, (2,5-dioxo-4-imidazolidinyl)- (=allantoin) of formula

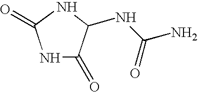

8. Furanones, like 3-Hydroxy4,5-dimethyl-2(5H)-furanone; 3-Hydroxy-4-methyl-5-ethyl-2(5H)-furanone;

9. Phenylacetaldehydes;

10. Benzaldehydes; like 4-hydroxybenzaldehyd and 3-methylbenzaldehyd;

11. 4-methoxycinnamaldehyde;

12. Isomeric Decene acid (C₁₀H18O₂);

13. Ascorbic acid and derivatives thereof, like 6-aycylascorbic acid-2-glucoside; sulfate, stearate or phosphate of ascorbic acid;

14. Salicylic acid derivatives like 6-[(8Z)-8-pentadecenyl]-salicylic acid; (anacardinacid-monoen) and 6-[(8Z, 11Z)-8, 11, 14-pentadecatrienyl] salicylic acid (anacardinacid-triene);

15. phenolic compounds like 3-[8(Z)-pentadecenyl]phenol or curuminphenolic compounds like curcumin or hydroxydiphenylether compounds like Triclosan or Diclosan;

16. Benzo[b]pyranderivative like [1]benzopyrano[5,4,3-cde][1]benzopyran-5, 10-dion, 2,3,7,8-tetrahydroxy- (7CI, 8CI, 9CI) (=Ellagic acid); 2′-hydroxy-2,4,4,7,4′-pentamethylflavan; 2′-Flavanol, 2,4,4,4′,7-pentamethyl-, acetate; 2-(3,4-dihydro-2,4,4,7-tetramethyl-2H-1-benzopyran-2-yl)-5-methylphenyl und (8β-giglycopyranosyl-7-hydroxy-5-methyl-2-(2-oxopropyl)-4H-1-benzopyran-4-on (Aloesin), of formula

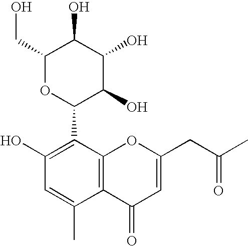

17. Bornyl- and cinnamate-derivatives, like 2-propenoic acid, 3-(4-hydroxyphenyl)-,1,7,7-trimethylbicyclo[2.2.1]hept-2-yl ester, endo-; 2-propenoic acid, 3-(4-methoxyphenyl)-, 1,7,7-trimethylbicyclo[2.2.1]hept-2-yl ester, endo-; 2-propenoic acid, 3-(4-hydroxyphenyl)-, 1-methyl-3-(2,2,6-trimethylcyclohexyl)propyl ester; 2-propenoic acid, 3-phenyl-, 1 -methyl-3-(2,2,6-trimethylcyclohexyl)propyl ester; 2-propenoic acid, 3-[4-(β-D-glucopyranosyloxy)phenyl]-, (1R,2S,4R)-1,7,7-trimethylbicyclo[2.2.1]hept-2-yl ester.

18. Azulenes and derivatives thereof like Guajazulen or Vetivazulen and Guaiol;

19. Cell messenger substances like cytokines; prostaglandines and peptide growth factors;

20. α-Hydroxy-carboxylic acids like α-hydroxypropionic acid, citric- and aconite acid

21. Compounds of formula

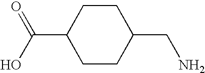

22. Compounds of formula

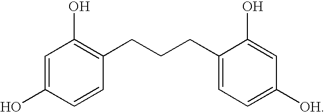

Preferably, the pigment regulator (d) is selected from kojic acid, arbutin, quercitin, aloesin, azelain acid, guaiol, and ellac acid.

The cosmetic or pharmaceutical preparations used for the present invention can be prepared by physically mixing components (a)-(d) with the adjuvant using customary methods, for example by simply stirring together the individual components, especially by making use of the dissolution properties of already known cosmetic UV absorbers, like octyl methoxy cinnamate, salicylic acid isooctyl ester, etc.

The cosmetic or pharmaceutical preparations may be, for example, creams, gels, lotions, alcoholic and aqueous/alcoholic solutions, emulsions, wax/fat compositions, stick preparations, powders or ointments. In addition to the above mentioned UV filters, the cosmetic or pharmaceutical preparations may contain further adjuvants.

As water- and oil-containing emulsions (e.g. W/O, O/W, O/W/O and W/O/W emulsions or microemulsions) the preparations contain, for example, from 0.1 to 30% by weight, preferably from 0.1 to 15% by weight and especially from 0.5 to 10% by weight, based on the total weight of the composition, of one or more UV absorbers, from 1 to 60% by weight, especially from 5 to 50% by weight and preferably from 10 to 35% by weight, based on the total weight of the composition, of at least one oil component, from 0 to 30% by weight, especially from 1 to 30% by weight und preferably from 4 to 20% by weight, based on the total weight of the composition, of at least one emulsifier, from 10 to 90% by weight, especially from 30 to 90% by weight, based on the total weight of the composition, of water, and from 0 to 88.9% by weight, especially from 1 to 50% by weight, of further cosmetically acceptable adjuvants.

The cosmetic or pharmaceutical compositions are useful agents for tanning prevention, skin wrinkling, anti-skinlifting and, together with pigment regulators (=component (d)), as skin lightening agents.

The cosmetic or pharmaceutical compositions/preparations according to the invention may also contain one or one more additional compounds like-fatty alcohols, esters of fatty acids, natural or synthetic triglycerides including glyceryl esters and derivatives, pearlescent waxes, hydrocarbon oils. silicones or siloxanes (organosubstituted polysiloxanes), fluorinated or perfluorinated oils, emulsifiers, super-fatting agents, surfactants, consistency regulators/thickeners and rheology modifiers, polymers. deodorising active ingredients, anti-dandruff agents, hvdrotropic agents, preservatives, antimicrobials, perfume oils, colourants, insect repellents or polymeric beads or hollow spheres as SPF enhancers

Cosmetic or Pharmaceutical Preparations

Cosmetic or pharmaceutical formulations are contained in a wide variety of cosmetic preparations, like skin-care preparations, cosmetic personal care preparations, light-protective preparations, skin-tanning preparations, depigmenting preparations, insect-repellents or preparations for cleansing and caring for blemished skin,

Presentation Forms

The final formulations listed may exist in a wide variety of presentation forms, for example:
in the form of liquid preparations as a W/O, O/W, O/W/O, W/O/W or PIT emulsion and all kinds of microemulsions,
in the form of a gel,
in the form of an oil, a cream, milk or lotion,
in the form of a powder, a lacquer, a tablet or make-up,
in the form of a stick,
in the form of a spray (spray with propellent gas or pump-action spray) or an aerosol,
in the form of a foam, or
in the form of a paste.

Of special importance as cosmetic preparations for the skin are light-protective preparations, such as sun milks, lotions, creams, oils, sunblocks or tropicals, pretanning preparations or after-sun preparations, also skin-tanning preparations, for example self-tanning creams. Of particular interest are sun protection creams, sun protection lotions, sun protection milk and sun protection preparations in the form of a spray.

The examples below serve to illustrate the invention without limiting it thereto. The cosmetic active substances are primarily given with their INCI name (INCI=Intemational Nomenclature of Cosmetic Ingredients).

### EXAMPLE

Materials and Methods

1. Skin Color Measurement:

Skin color was measured in the L*a*b* colour by means of a Minolta CM-508i®) Chromameter).

2. Biophysical Skin Parameters

Skin moisture was assessed with a Corneometer® and skin elasticity and tension by a Cutometer®) (both from Courage & Khazaga, Kön, Germany).

Skin smoothness and skin wrinkles were measured by contact-free profilometry, Leica, Quantimet 6® (Leica Heidelberg, Germany).

3. Testpanel

a. Asian Volunteers

10 Volunteers of originating from S-E-Asia and with a skinlightness L* between 65.4 and 40.8 were recruited following COLIPA-Guidelines.

b. Indian Volunteers

10 Volunteers of Indian origin with a skinlightness L* between 41.1. and 33.6 were recruited following COLIPA-Guidelines.

c. White Caucasian Volunteers

10 Volunteers of European origin with a skin lightness L* between 68.8 and 56.2 were recruited following COLIPA-Guidelines.

4. Volunteers and Measurements

The influence of extensive sun exposure on skin pigmentation and the protective effects of day care cosmetics were directly measured on the back of volunteers:

40 and 20 minutes prior to irradiation volunteers treated on the back with 2 mg/cm² of cream formulation.

Then tey were irradiated with simulated solar light for 12 weeks, 3-times a week with a dose≦1 MED (Minimal erythema dose; MED adjusted monthly).

The skin of the volunteers was assessed prior to the test and monthly prior to cream application and irradiation.

### Example 1

Tanning Prevention

The influence of extensive sun exposure on skin pigmentation and the protective effects of a day cream containing

Aqua (up to 100%);

| | |
|---|---|
| 5% | Caprylic/Capric Triglyceride |
| 5% | C₁₂-C₁₅ Alkyl Benzoate |
| 5% | Cetearyl Isononanoate |
| 3% | Glyceryl Stearate |
| 3% | Glycerin |
| 2% | Potassium Cetyl Phosphate |
| 1% | Cetyl Alcohol |
| 1% | Cetyl Stearate |
| 1% | Phenoxyethanol/Parabenes |
| 0.5% | Steareth-10 Allyl Ether/Acrylates Copolymer and |
| 5% | of the compound of formula (3) |

was directly measured on the back of Asian volunteers.

The skin of the volunteers was assessed prior to the test and monthly prior to cream application and irradiation for skin color by a Minolta Colorimeter 508i.

As documented, skin tanning could be markedly prevented by the day cream

Tanning prevention by daycreams (10 Asian volunteers)

| | Without protection | 5% of the compound of |
|---|---|---|
| L*/a*/b* 3xw < 1MED | [L*/a*/b*] | formula (3) |
| Start | 52.3/21.0/22.0 | 53.3/22.5/22.5 |
| After 4 weeks | 44.8/27.3/26.5 | 50.5/23.1/22.6 |
| After 8 weeks | 42.0/30.8/28.4 | 48.8/22.6/23.3 |
| After 12 weeks | 38.8/30.1/27.9 | 47.7/21.5/24.6 |

### Example 2

Prevention of Skin Wrinkling by UV-A Absorbing Day Cream

Skin smoothness and skin wrinkles were assessed on the back of 10 Indian volunteers prior to the test and monthly prior to cream application and irradiation by a contact-free profilometry, Leica, Quantimet 600® (Leica Heidelberg, Germany).

UV Day Cream

| | | % w/w |
|---|---|---|
| | INCI-Name | (as supplied) |
| Part A | Cetyl Phosphate | 1.75 |
| | C₁₂-C₁₅ Alkyl Benzoate | 4.00 |
| | Cetearyl Alcohol/PEG-20 Stearate | 2.00 |
| | Ethoxydiglycol Oleate | 2.00 |
| | Stearic Acid | 1.50 |
| | Ethylhexyl Triazone (Uvinul T150) | 2.00 |
| | Isononyl Isononanoate | 2.00 |
| | Bis-ethylhexyloxyphenol methoxyphenyl Triazine | 1.00 |
| | (Tinosorb S) | |
| Part B | Aqua | qs to 100 |
| | Xanthan Gum | 0.35 |
| | Compound of formula (3) (Uvinul A Plus) | 2.00 |
| | Disodium EDTA | 0.20 |
| | Propylene Glycol | 2.00 |
| | Diazolidinyl Urea (and) Methylparaben (and) | 0.70 |
| | Propylparaben (and) Propylene Glycol | |
| | Glycerin | 1.50 |
| Part C | Cyclopentasiloxane (and) Dimethiconol | 1.00 |
| | Ethoxydiglycol | 3.00 |
| | Dimethicone | 2.00 |
| Part D | Triethanolamine | qs |

Manufacturing Instruction:

Part A by is prepared by incorporating all ingredients, then stirred under moderate speed and heated to 75° C. Part B s prepared and heated to 75° C. At this temperature part B is poured into part A under progressive stirring speed. Then the mixture is homogenized (30sec., 15000 rpm ). At a temperature<55° C. the ingredients of part C are incorporated. The mixture is cooled down under moderate stirring, then the pH is checked and adjusted with triethanolamine.

Prevention of wrinkles and loss of smoothness on 10 Indian volunteers

| Without | | | | |
|---|---|---|---|---|
| protection | | | | |
| L*/a*/b*3x | | UV Day Cream | | UV Day Cream |
| w < 1MED | wrinkles | Formulation | smoothness | Formulation |
| start | Control | Cream | Control | Cream |
| 4 weeks | 4.7% | −7.8% | −7.7% | 7.5% |
| 8 weeks | 7.8% | −9.0% | −8.7%( | 10.2% |
| 12 weeks | 10.5% | −12.8% | −8.1% | 15.4% |

### Example 3

Retention of Moisturing Activity of Day Cream Thanks to UV-Absorber Measured on 12 Caucasian Volunteers

Skin moisture was assessed with a Corneometer® prior to the test and monthly prior to irradiation.

Skin moisture in % of start values 10 Caucasian volunteers

| Start | No cream | Placebo cream | Cream with UV-Absorber |
|---|---|---|---|
| 4 weeks | −16.5% | 12.7% | 31.8 |
| 8 weeks | −19.1% | 14.6% | 46.4% |
| 12 weeks | −21.3% | 16.2% | 45.8% |

As can besen from the results of Table 3, the moisturing activity is significantly enhenced with the UV absorber containing cream as sued in the present invetion.

Day cream Composition Formulation A (=Placebo Cream):

| | |
|---|---|
| 4% | Cetaryl isonoate, C12-15 Alkyl Benzoate, |
| 3.5% | Dioctyl Ether, |
| 2% | Cetearyl Alcohol/PEG-20 Stearate, |
| 2% | Isohexadecane, |
| 2% | Ethoxydiglycol Oleate, |
| 2% | Cetyl Phosphate, |
| 1% | Cetyl Alcohol, |
| 0.7% | Steareth-10 Allyl Ether/Acrylates Copolymer) and |
| 1% | Ethylhexyl Triazone |
| 0.75% | Benzophenone-3 and |
| 1.5% | Ethylhexyl Methoxycinnamate |
| Ad 100% | water. |

Day cream Composition Formulation B:

| | |
|---|---|
| 4% | Cetaryl isonoate, C₁₂₋₁₅ Alkyl Benzoate, |
| 3.5% | Dioctyl Ether, |
| 2% | Cetearyl Alcohol/PEG-20 Stearate, |
| 2% | Isohexadecane, |
| 2% | Ethoxydiglycol Oleate, |
| 2% | Cetyl Phosphate, |
| 1% | Cetyl Alcohol, |
| 0.7% | Steareth-10 Allyl Ether/Acrylates Copolymer and |
| 0.5% | Ethylhexyl Triazone |
| 1.25% | of the compound of formula (3); and |
| 2% | 2,2′-Methylene-bis-[6-(2H-benzotriazol-2-yl)-4-(1,1,3,3- |
| | tetramethylbutyl)-phenol]; (Tinosorb M). |
| Ad 100% | Water, |

1. A cosmetic or pharmaceutical composition, useful as an organic UV filter for preventing the tanning of human skin, comprising
(a) UV filters selected from the compounds of formula 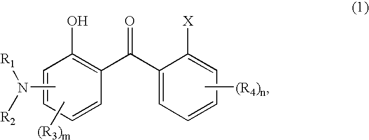
wherein
R₁ and R₂, independently from each other are hydrogen; C₁-C₂₀alkyl; C₂ -C₁₀alkenyl; C₃-C₁₀cycloalkyl; C₃-C₁₀cycloalkenyl; or R₁ and R₂ together with the nitrogen atom to which they are bonded can form a 5- or 6-membered ring;
R₃ and R₄, independently from each other are C₁-C₂₀alkyl; C₂-C₁₀alkenyl; C₃-C₁₀-cycloalkyl; C₃-C₁₀cycloalkenyl; C₁-C₁₂alkoxy; C₁-C₂₀alkoxycarbonyl; C₁-C₁₂alkylamino; C₁-C₁₂dialkylamino; C₆-C₁₀aryl; C₆-C₁₀heteroaryl, optionally substituted, substituents which confer solubility in water, chosen from the group consisting of a nitrile group, carboxylate, sulfonate or ammonium radicals;
X is hydrogen; COOR₅; CONR₆R₇;
R₅, R₆ and R₇, independently from each other are hydrogen; C₁-C₂₀alkyl; C₂-C₁₀alkenyl; C₃-C₁₀cycloalkyl; C₃-C₁₀cycloalkenyl; or (Y—O)ₒ-Z-aryl;
Y is —(CH₂)₂—; —(CH₂)₃; —(CH₂)₄—; —CH(CH₃)—CH₂—;
Z is —CH₂—CH₃; —CH₂—CH₂—CH₃; —CH₂—CH₂—CH₂—CH₃; —CH(CH₃)—CH₃;
m is a number from 0 to 3;
n is a number from 0 to 4; and
o is a number from 1 to 20

2. A composition according to claim 1, wherein the UV filters (a) correspond to formula 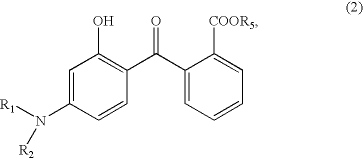
wherein
R₁ and R₂ independently from each other are hydrogen, C₁-C₁₂alkyl; or R₁ and R.₂ together with the nitrogen atom to which they are bonded form a 5- or 6-membered ring; and
R₅ is hydrogen; C₁-Cl₂alkyl; or C₃-C₆cycloalkyl.

3. A composition according to claim 1, wherein
R₁ and R₂, independently from each other are C₁-C₅alkyl; and
R₅ is C₁-C₁₂alkyl.

4. A composition according to claim 1, wherein
R₁ and R₂ have the same meaning.

5. A composition according to claim 1, wherein the UV filters (a) correspond to formula

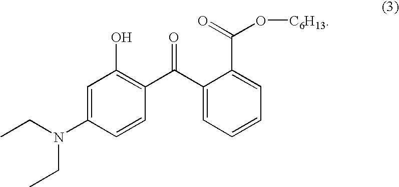

6. A composition according to claim 1, which contains
(b) at least one additional UV filter.

7. A composition according to claim 6 wherein the additional UV filters (b) are selected from p-aminobenzoic acid derivatives, salicylic acid derivatives, benzophenone derivatives different from those of formula (1), dibenzoylmethane derivatives, diphenylacrylates, 3-imidazol-4-ylacrylic acid and esters; benzofuran derivatives, polymeric UV absorbers, cinnamic acid derivatives, camphor derivatives, hydroxyphenyltriazine compounds, benzotriazole compounds, trianilino-s-triazine derivatives, 2-phenylbenzimidazole-5-sulfonic acid and salts thereof, menthyl o-aminobenzoates, physical coated or non-coated sunscreens, perfluoroalcohol phosphate, aminohydroxy-benzophenone derivatives and phenyl-benzimidazole derivatives.

8. A composition according to claim 1, which contains
(c) a pharmaceutical or cosmetic active ingredient.

9. A composition according to claim 8, wherein the pharmaceutical or cosmetic active ingredient (c) is an antioxidant.

10. A composition according to claim 1, which contains
(d) a pigment regulator agent.

11. A composition according to claim 10, wherein the pigment regulator agent (d) is a skin lightening agent and is selected from γ-pyron derivates, hydroquinone, resorcin derivatives; glycines; alkyl dicarboxylic acids;, 1,2-dihydroxyphenylderivatives; urea, furanones, phenylacetaldehydes; benzaldehydes; 4-methoxycinnamaldehydes; isomeic decene acids; ascorbic acid and derivatives thereof; salicylic acid derivatives thereof; phenolic compounds; benzo[b]pyran derivatives, bornyl- and cinnamate derivates, azulenes and derivates thereof; α-hydroxy-carboxylic acids; the compound of formula 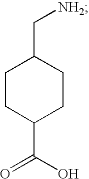
and the compound of formula

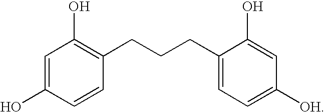

12. A composition according to claim 11, wherein the agent (d) is selected from kojic acid, arbutin, quercitin, aloesin, azelain acid, guaiol, and ellag acid.

13. A cosmetic or pharmaceutical composition according to claim 1 further comprising
(b) at least one additional UV filter and
(c) a pharmaceutical or cosmetic active ingredient.

14. A cosmetic or pharmaceutical composition according to claim 1 further comprising
(b) at least one additional UV filter and
(d) a pigment regulator agent.

15. A cosmetic or pharmaceutical composition according to claim 1 further comprising
(c) a pharmaceutical or cosmetic active ingredient and
(d) a pigment regulator agent.
